(19) United States
(12) Patent Application Publication
(10) Pub. No.: US 2009/0213211 A1
(43) Pub. Date: Aug. 27, 2009

(54) Method and Device for Reducing the Fixed Pattern Noise of a Digital Image

(75) Inventors: Lex Bayer, Palo Alto, CA (US); Michael Stewart, Menlo Park, CA (US)

Correspondence Address: SQUIRE, SANDERS & DEMPSEY L.L.P., 1 MARITIME PLAZA, SUITE 300, SAN FRANCISCO, CA, 94111, US

(73) Assignee: Avantis Medical Systems, Inc., Sunnyvale, CA (US)

(21) Appl. No.: 12/251,406

(22) Filed: Oct. 14, 2008

Related U.S. Application Data
(60) Provisional application No. 60/979,368, filed on Oct. 11, 2007.

(51) Int. Cl. H04N 7/18; G06K 9/40; H04N 5/217; H04N 5/76

(52) U.S. Cl. 348/65; 382/260; 348/241; 348/231.99; 348/E05.078; 348/E07.085

(57) ABSTRACT

A method or a device that reduces fixed pattern noise in an image captured by a digital image device and adjusts the reduction based on the level of FPN, preferably on an area-by-area basis or on a pixel-by-pixel basis.

This application claims the benefit of U.S. Provisional Patent Application No. 60/979,368, filed Oct. 11, 2007, the entire disclosure of which is incorporated herein by reference.

### FIELD OF THE INVENTION

The present invention relates to a method for reducing the fixed pattern noise of a digital image and a device for reducing the fixed pattern noise of a digital image.

### BACKGROUND OF THE INVENTION

Digital imaging devices have a variety of applications. For example, they are used in endoscopic devices for medical procedures or for inspecting small pipes or for remote monitoring. One example of such endoscopic devices is an endoscope having a retrograde-viewing auxiliary imaging device, which is being developed by Avantis Medical Systems, Inc. of Sunnyvale, Calif.

There are various types of digital imaging devices. On example is a digital imaging device using complementary metal oxide semiconductor (CMOS) technology. During operation, each pixel of the device generates a charge, the charges from all pixels are used to generate an image. Each charge includes three portions. A first portion of each charge is related to the photon rate. In other words, when a CMOS pixel in an imaging device is exposed to light emitted from an image, photons in the light strike the pixel, generating this first portion of the charge, the magnitude of which is related to the photon rate. A second portion of each charge is due to inaccuracies and inconsistencies inherent in each pixel, such as those resulting from the variations in manufacturing and sensor materials. The inaccuracies and inconsistencies vary from pixel to pixel, causing this portion of the charge to vary from pixel to pixel. This second portion exists even when there is no light reaching the pixel. The third portion of each charge is a function of the location of the pixel within the imaging device and the operating condition of the pixel, such as the operating temperature and exposure parameters such as brightness. This third portion is often negative. For example, an increase in photo rate results in a reduction in pixel charge. Needless to say, the third portion also varies from pixel to pixel.

The second and third portions of the pixel charges distort the true image signals and give rise to fixed pattern noise (FPN) in the image. FPN appears as snow-like dots on a captured image and reduces the image's quality. It is highly desirable to remove the FPN from the sensed image to improve the quality of the image.

Cancellation of FPN can be achieved by capturing a “dark image” when no light is reaching the CMOS imaging device. The dark image data are presumed to represent FPN and subtracted from the sensed image data to produce “corrected” image data. However, this method does not take into consideration the third portion of the pixel charge. In other words, the level of FPN in an area of the image is not only a function of inherent pixel parameters, which this method captures, but also a function of the operating parameters, such as the brightness of the image in the area, which this method does not capture. Therefore, this conventional method of using “dark image” data to cancel FPN produces the effect that the brighter areas of the image with low levels of FPN are overcompensated, resulting in the degradation of the image in those areas.

Medical endoscopes often produce video images which have rapidly changing dark and bright areas. Although the FPN in the dark areas is adequately compensated by conventional FPN reduction methods, bright areas of the image tend to have low levels of FPN and are overcompensated by conventional FPN reduction methods, resulting in a degradation of the image in the bright areas. Therefore, the conventional methods of cancelling FPN may improve the image quality in the dark areas of an image while degrading the image quality in the bright areas of the image.

### SUMMARY OF THE INVENTION

One aspect of the present invention is directed to a method or a device that reduces FPN in an image captured by a digital imaging device and adjusts the reduction based on the level of FPN, preferably on an area-by-area basis or on a pixel-by-pixel basis. A preferred embodiment of the present invention uses the brightness of each area or pixel and the gain of the image to determine the level of FPN and then subtracts the determined level of FPN from the image signals measured in the area or for the pixel. Generally, however, other operating parameters, such as the operating temperature, the captured light's color composition, and the imaging sensor's voltage level, may also be used to determine the level of FPN in an area or for a pixel.

In one embodiment, a baseline FPN is determined from a dark image or an image taken under a given light condition either periodically or initially at the manufacturer. Then the “actual” FPN is determined based on the baseline FPN and on one or more of the “relevant variables,” which are defined as the variables that affect the FPN level of the area or pixel. These relevant variables include, but are not limited to, the brightness and color composition of the area or pixel, the operating temperature, the imaging sensor's voltage level and the gain of the image. The “actual” FPN is then subtracted from the area's image signals or the pixel's image signal. This results in an improved image with reduced degradation in the bright areas of the image. This may be done for every frame or a selected number of frames in the case of a video image signal.

According to one aspect of the invention, a method for reducing a digital image's fixed pattern noise includes determining the amount of FPN in a digital image taken by a digital imaging device as a function of at least one of brightness level, operating temperature, and gain value of the image on an area-by-area basis or on a pixel-by-pixel basis; and modifying the digital image by the determined amount of FPN on an area-by-area basis or on a pixel-by-pixel basis.

In one embodiment according to this aspect of the invention, the step of determining includes determining the amount of FPN as a function of only the brightness level of the image on an area-by-area basis or on a pixel-by-pixel basis.

In one other embodiment according to this aspect of the invention, the step of determining includes determining the amount of FPN as a function of only the brightness level and gain value of the image on an area-by-area basis or on a pixel-by-pixel basis.

In another embodiment according to this aspect of the invention, the step of determining includes determining the amount of FPN as a function of only the gain value of the image on an area-by-area basis or on a pixel-by-pixel basis.

In still another embodiment according to this aspect of the invention, the step of determining includes determining the amount of FPN as a function of the brightness level, operating temperature, and gain value of the image on an area-by-area basis or on a pixel-by-pixel basis.

In yet another embodiment according to this aspect of the invention, the step of determining includes obtaining a dark FPN image from the imaging device with the imaging device in a dark environment.

In yet still another embodiment according to this aspect of the invention, the step of determining includes determining a subtraction factor for each area or pixel using a look-up table having the subtraction factor as an output and the at least one of brightness level, operating temperature, and gain value of the image as one or more inputs.

In a further embodiment according to this aspect of the invention, the step of determining includes determining the amount of FPN in the digital image by using the subtraction factor for each area or pixel to reduce the dark FPN value for this area or pixel.

In a still further embodiment according to this aspect of the invention, the step of determining includes determining a subtraction factor for each area or pixel using an equation having the subtraction factor at an independent variable and the at least one of brightness level, operating temperature, and gain value of the image as one or more dependent variable.

In a yet further embodiment according to this aspect of the invention, the step of determining includes determining the amount of FPN in the digital image by using the subtraction factor for each area or pixel to reduce the dark FPN value for this area or pixel.

In a still yet further embodiment according to this aspect of the invention, the step of obtaining a dark FPN image includes obtaining the dark FPN image as part of an initial factory calibration.

In another embodiment according to this aspect of the invention, the step of obtaining a dark FPN image includes obtaining periodically during the life of the imaging device.

In a further embodiment according to this aspect of the invention, the digital image is in YUV format, the method further comprising determining the brightness level from the luma component of the YUV format digital image.

In a still further embodiment according to this aspect of the invention, the digital image is in RGB format, the method further comprising converting the RGB format digital image to a YUV format digital image, and determining the brightness level from the luma component of the YUV format digital image.

In accordance with another aspect of the invention, a device for reducing a digital image's fixed pattern noise includes an input for receiving a digital image from a digital imaging device; an output for sending a modified digital image to a display device; a processor that includes one or more circuits and/or software for processing the digital image. The processor determines the amount of FPN in the digital image as a function of at least one of brightness level, operating temperature, and gain value of the image on an area-by-area basis or on a pixel-by-pixel basis and modifies the digital image by the determined amount of FPN on an area-by-area basis or on a pixel-by-pixel basis.

In one embodiment according to this aspect of the invention, the at least one of brightness level, operating temperature, and gain value of the image consists of the brightness level of the image.

In one other embodiment according to this aspect of the invention, the at least one of brightness level, operating temperature, and gain value of the image consists of the brightness level and gain value of the image.

In another embodiment according to this aspect of the invention, the at least one of brightness level, operating temperature, and gain value of the image consists of the gain value of the image.

In still another embodiment according to this aspect of the invention, the at least one of brightness level, operating temperature, and gain value of the image includes the brightness level, operating temperature, and gain value of the image.

In yet another embodiment according to this aspect of the invention, the processor determines the amount of FPN in the digital image by way of obtaining a dark FPN image from the imaging device with the imaging device in a dark environment.

In still yet another embodiment according to this aspect of the invention, the processor determines the amount of FPN in the digital image by way of determining a subtraction factor for each area or pixel using a look-up table having the subtraction factor as an output and the at least one of brightness level, operating temperature, and gain value of the image as one or more inputs.

In a further embodiment according to this aspect of the invention, the processor determines the amount of FPN in the digital image by way of using the subtraction factor for each area or pixel to reduce the dark FPN value for this area or pixel.

In a still further embodiment according to this aspect of the invention, the processor determines the amount of FPN in the digital image by way of determining a subtraction factor for each area or pixel using an equation having the subtraction factor at an independent variable and the at least one of brightness level, operating temperature, and gain value of the image as one or more dependent variable.

In a yet further embodiment according to this aspect of the invention, the processor determines the amount of FPN in the digital image by way of using the subtraction factor for each area or pixel to reduce the dark FPN value for this area or pixel.

In a still yet further embodiment according to this aspect of the invention, the processor obtains the dark FPN image as part of an initial factory calibration.

In another embodiment according to this aspect of the invention, the processor obtains the dark FPN image periodically during the life of the imaging device.

In still another embodiment according to this aspect of the invention, the digital image is in YUV format, and the processor determines the brightness level from the luma component of the YUV format digital image.

In yet another embodiment according to this aspect of the invention, the digital image is in RGB format, and the processor converts the RGB format digital image to a YUV format digital image and determines the brightness level from the luma component of the YUV format digital image.

In accordance with still another aspect of the invention, an endoscope system includes the device of claim 15; an endoscope including the digital imaging device and being connected to the input of the device; and a displace device that is connected to the output of the device to receive and display the modified digital image.

In one embodiment according to this aspect of the invention, the digital imaging device is a retrograde-viewing auxiliary imaging device.

In accordance with yet another aspect of the invention, a method for sharpening a digital image includes determining the amount of sharpening needed to sharpen a digital image taken by a digital imaging device as a function of at least one of brightness level, operating temperature, and gain value of the image on an area-by-area basis or on a pixel-by-pixel basis; and sharpening the digital image by the determined amount of sharpening on an area-by-area basis or on a pixel-by-pixel basis.

In accordance with still another aspect of the invention, a device for sharpening a digital image includes an input for receiving a digital image from a digital imaging device; an output for sending a sharpened digital image to a display device; a processor that includes one or more circuits and/or software for shapening the digital image. The processor determines the amount of sharpening needed to sharpen the digital image as a function of at least one of brightness level, operating temperature, and gain value of the image on an area-by-area basis or on a pixel-by-pixel basis and sharpens the digital image by the determined amount of sharpening on an area-by-area basis or on a pixel-by-pixel basis.

For easy of description, the present invention will be described in the context of the retrograde-viewing auxiliary imaging device of Avantis Medical Systems, Inc. of Sunnyvale, Calif. However, this is meant to limit the scope of the invention, which has broader applications in other fields, such as endoscopy in general.

### BRIEF DESCRIPTION OF THE DRAWINGS

FIG. 1 shows a perspective view of an endoscope with an imaging assembly according to one embodiment of the present invention.

FIG. 2 shows a perspective view of the distal end of an insertion tube of the endoscope of FIG. 1.

FIG. 3 shows a perspective view of the imaging assembly shown in FIG. 1.

FIG. 4 shows a perspective view of the distal ends of the endoscope and imaging assembly of FIG. 1.

FIG. 5 shows a block diagram illustrating an endoscope system of the present invention.

FIG. 6 shows a block diagram illustrating a procedure of the present invention.

FIG. 7 shows images generated by the procedure illustrated in FIG. 6.

FIG. 8 shows a block diagram illustrating an embodiment of the present invention that allows for dynamic sharpening.

### DETAILED DESCRIPTION OF EMBODIMENTS OF THE INVENTION

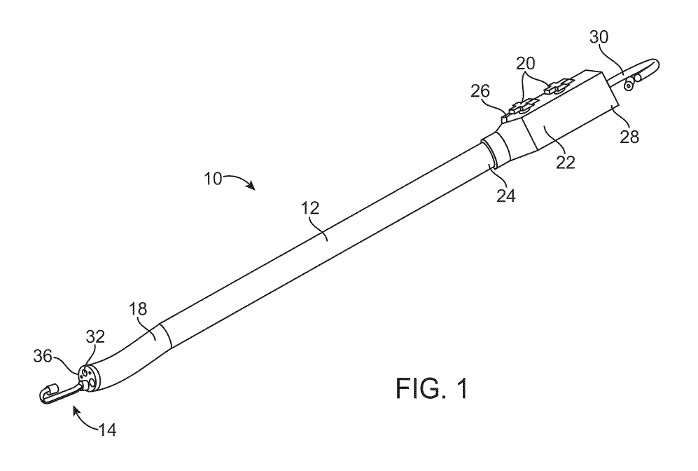

FIG. 1 illustrates an exemplary endoscope 10 of the present invention. This endoscope 10 can be used in a variety of medical procedures in which imaging of a body tissue, organ, cavity or lumen is required. The types of procedures include, for example, anoscopy, arthroscopy, bronchoscopy, colonoscopy, cystoscopy, EGD, laparoscopy, and sigmoidoscopy.

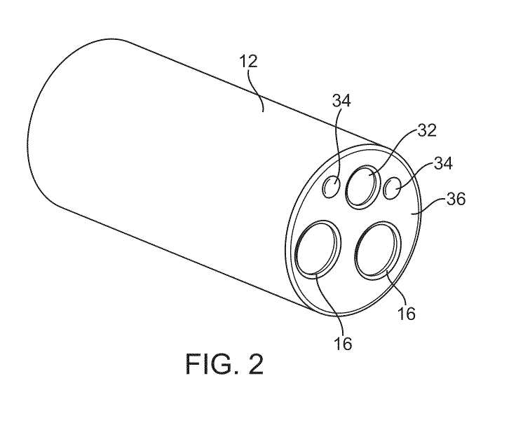

The endoscope 10 of FIG. 1 includes an insertion tube 12 and an imaging assembly 14, a section of which is housed inside the insertion tube 12. As shown in FIG. 2, the insertion tube 12 has two longitudinal channels 16. In general, however, the insertion tube 12 may have any number of longitudinal channels. An instrument can reach the body cavity through one of the channels 16 to perform any desired procedures, such as to take samples of suspicious tissues or to perform other surgical procedures such as polypectomy. The instruments may be, for example, a retractable needle for drug injection, hydraulically actuated scissors, clamps, grasping tools, electrocoagulation systems, ultrasound transducers, electrical sensors, heating elements, laser mechanisms and other ablation means. In some embodiments, one of the channels can be used to supply a washing liquid such as water for washing. Another or the same channel may be used to supply a gas, such as CO₂ or air into the organ. The channels 16 may also be used to extract fluids or inject fluids, such as a drug in a liquid carrier, into the body. Various biopsy, drug delivery, and other diagnostic and therapeutic devices may also be inserted via the channels 16 to perform specific functions.

The insertion tube 12 preferably is steerable or has a steerable distal end region 18 as shown in FIG. 1. The length of the distal end region 18 may be any suitable fraction of the length of the insertion tube 12, such as one half, one third, one fourth, one sixth, one tenth, or one twentieth. The insertion tube 12 may have control cables (not shown) for the manipulation of the insertion tube 12. Preferably, the control cables are symmetrically positioned within the insertion tube 12 and extend along the length of the insertion tube 12. The control cables may be anchored at or near the distal end 36 of the insertion tube 12. Each of the control cables may be a Bowden cable, which includes a wire contained in a flexible overlying hollow tube. The wires of the Bowden cables are attached to controls 20 in the handle 22. Using the controls 20, the wires can be pulled to bend the distal end region 18 of the insertion tube 12 in a given direction. The Bowden cables can be used to articulate the distal end region 18 of the insertion tube 12 in different directions.

As shown in FIG. 1, the endoscope 10 may also include a control handle 22 connected to the proximal end 24 of the insertion tube 12. Preferably, the control handle 22 has one or more ports and/or valves (not shown) for controlling access to the channels 16 of the insertion tube 12. The ports and/or valves can be air or water valves, suction valves, instrumentation ports, and suction/instrumentation ports. As shown in FIG. 1, the control handle 22 may additionally include buttons 26 for taking pictures with an imaging device on the insertion tube 12, the imaging assembly 14, or both. The proximal end 28 of the control handle 22 may include an accessory outlet 30 (FIG. 1) that provides fluid communication between the air, water and suction channels and the pumps and related accessories. The same outlet 30 or a different outlet can be used for electrical lines to light and imaging components at the distal end of the endoscope 10.

As shown in FIG. 2, the endoscope 10 may further include an imaging device 32 and light sources 34, both of which are disposed at the distal end 36 of the insertion tube 12. The imaging device 32 may include, for example, a lens, single chip sensor, multiple chip sensor or fiber optic implemented devices. The imaging device 32, in electrical communication with a processor and/or monitor, may provide still images or recorded or live video images. The light sources 34 preferably are equidistant from the imaging device 32 to provide even illumination. The intensity of each light source 34 can be adjusted to achieve optimum imaging. The circuits for the imaging device 32 and light sources 34 may be incorporated into a printed circuit board (PCB).

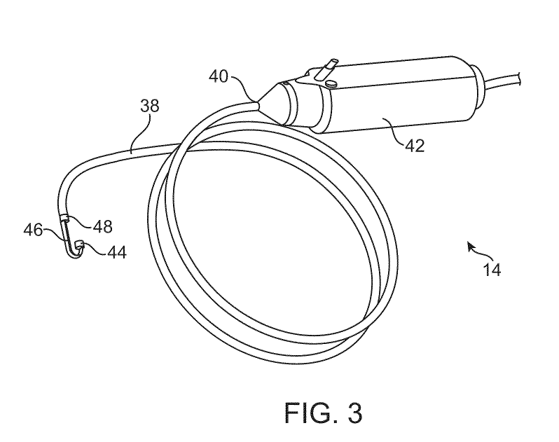

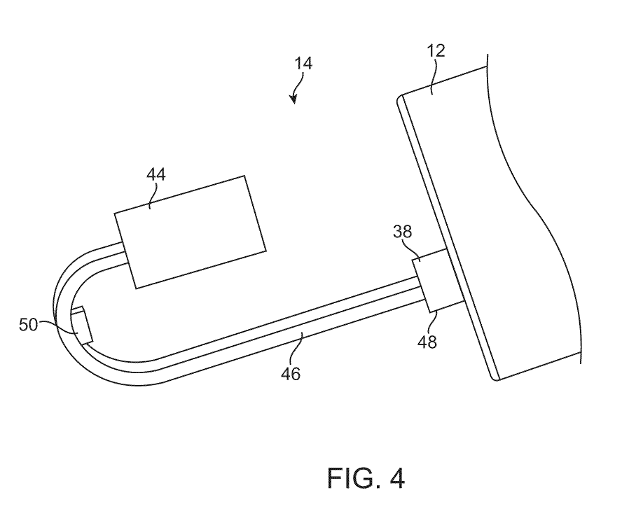

As shown in FIGS. 3 and 4, the imaging assembly 14 may include a tubular body 38, a handle 42 connected to the proximal end 40 of the tubular body 38, an auxiliary imaging device 44, a link 46 that provides physical and/or electrical connection between the auxiliary imaging device 44 to the distal end 48 of the tubular body 38, and an auxiliary light source 50 (FIG. 4). The auxiliary light source 50 may be an LED device.

As shown in FIG. 4, the imaging assembly 14 of the endoscope 10 is used to provide an auxiliary imaging device at the distal end of the insertion tube 12. To this end, the imaging assembly 14 is placed inside one of the channels 16 of the endoscope's insertion tube 12 with its auxiliary imaging device 44 disposed beyond the distal end 36 of the insertion tube 12. This can be accomplished by first inserting the distal end of the imaging assembly 14 into the insertion tube's channel 16 from the endoscope's handle 18 and then pushing the imaging assembly 14 further into the assembly 14 until the auxiliary imaging device 44 and link 46 of the imaging assembly 14 are positioned outside the distal end 36 of the insertion tube 12 as shown in FIG. 4.

Each of the main and auxiliary imaging devices 32, 44 may be an electronic device which converts light incident on photosensitive semiconductor elements into electrical signals. The imaging device may detect either color or black-and-white images. The signals from the imaging device can be digitized and used to reproduce an image that is incident on the imaging device. Preferably, the main imaging device 32 is a CCD imaging device, and the auxiliary imaging device 44 is a CMOS imaging device, either imaging device can be a CCD imaging device or a CMOS imaging device.

When the imaging assembly 14 is properly installed in the insertion tube 12, the auxiliary imaging device 44 of the imaging assembly 14 preferably faces backwards towards the main imaging device 32 as illustrated in FIG. 4. The auxiliary imaging device 44 may be oriented so that the auxiliary imaging device 44 and the main imaging device 32 have adjacent or overlapping viewing areas. Alternatively, the auxiliary imaging device 44 may be oriented so that the auxiliary imaging device 44 and the main imaging device 32 simultaneously provide different views of the same area. Preferably, the auxiliary imaging device 44 provides a retrograde view of the area, while the main imaging device 32 provides a front view of the area. However, the auxiliary imaging device 44 could be oriented in other directions to provide other views, including views that are substantially parallel to the axis of the main imaging device 32.

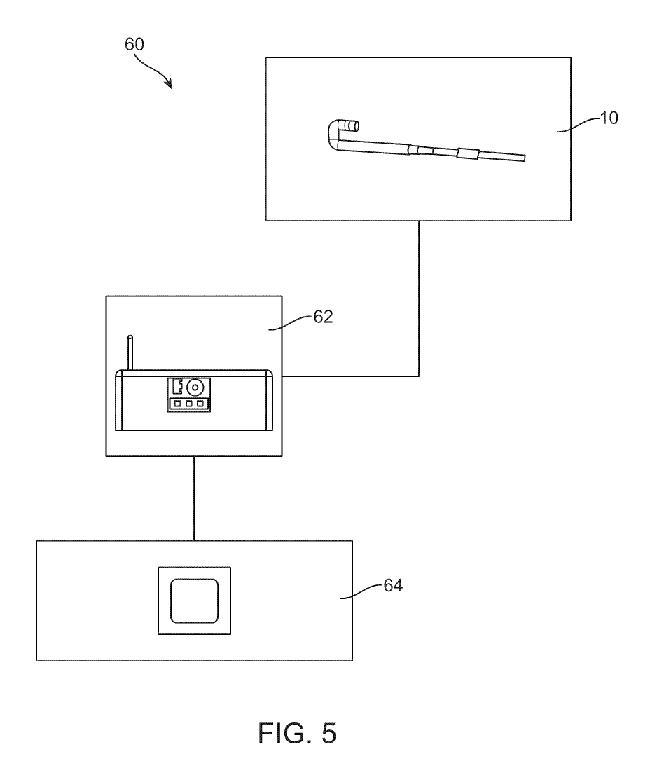

As shown in FIG. 4, the link 46 connects the auxiliary imaging device 44 to the distal end 48 of the tubular body 38. Preferably, the link 46 is a flexible link that is at least partially made from a flexible shape memory material that substantially tends to return to its original shape after deformation. Shape memory materials are well known and include shape memory alloys and shape memory polymers. A suitable flexible shape memory material is a shape memory alloy such as nitinol. The flexible link 46 is straightened to allow the distal end of the imaging assembly 14 to be inserted into the proximal end of assembly 14 of the insertion tube 12 and then pushed towards the distal end 36 of the insertion tube 12. When the auxiliary imaging device 44 and flexible link 46 are pushed sufficiently out of the distal end 36 of the insertion tube 12, the flexible link 46 resumes its natural bent configuration as shown in FIG. 3. The natural configuration of the flexible link 46 is the configuration of the flexible link 46 when the flexible link 46 is not subject to any force or stress. When the flexible link 46 resumes its natural bent configuration, the auxiliary imaging device 44 faces substantially back towards the distal end 36 of the insertion tube 12 as shown in FIG. 5.

In the illustrated embodiment, the auxiliary light source 50 of the imaging assembly 14 is placed on the flexible link 46, in particular on the curved concave portion of the flexible link 46. The auxiliary light source 50 provides illumination for the auxiliary imaging device 44 and may face substantially the same direction as the auxiliary imaging device 44 as shown in FIG. 4.

An endoscope of the present invention, such as the endoscope 10 shown in FIG. 1, may be part of an endoscope system 60 that may also include a video processor 62 and a display device 64, as shown in FIG. 5. In the preferred embodiment shown in FIG. 5, the video processor 62 is connected to the main and/or auxiliary imaging devices 32, 44 of the endoscope 10 to receive image data and to process the image data and transmit the processed image data to the display device 64. The connection between the video processor 62 and the imaging device 32, 44 can be either wireless or wired. The video processor 62 may also transmit power and control commands to the main and/or auxiliary imaging devices 32, 44 and receive control settings from the main and/or auxiliary imaging devices 32, 44.

In one preferred embodiment of the invention, the video processor 62 may have algorithm and/or one or more circuits for reducing FPN in the video output image of the main imaging device 32 and/or in the video output image of the auxiliary imaging device 44.

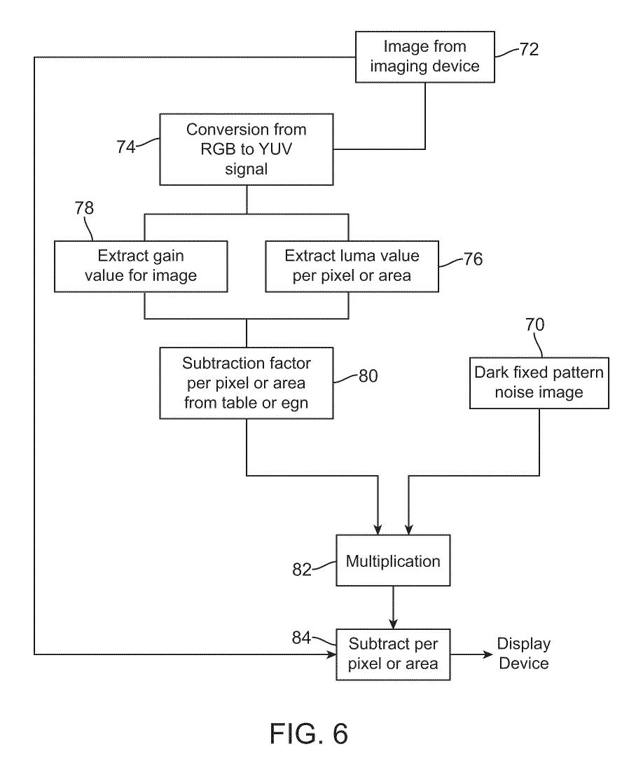

As illustrated in FIG. 6, as a first step 70 of the procedure for reducing FPN, an FPN image is acquired by the imaging device 32, 44 with the imaging device 32, 44 in a dark environment devoid of light. This can be done as part of an initial factory calibration or periodically during the life of the imaging device 32, 44, such as every second during operation or at the beginning of each operation. FPN is at its highest level when there is no light in the field of view, which requires the sensor gain to be at the maximum. This serves as a baseline for FPN reduction. This dark FPN image is then stored in the memory of the imaging device 32, 44 such as EEPROM or in the memory of the video processor 62.

In the second step 72, a digital image is sent from the imaging device 32, 44 to the video processor 62.

In the third step 74, if the output image of the imaging device 32, 44 is an RGB signal, the RGB signal is converted to a YUV signal, which has one brightness component and two color components. If the output image of the imaging device 32, 44 is a YUV signal, the conversion is unnecessary.

In the fourth step 76, from the YUV signal, the luma or brightness component is analyzed and a brightness value is obtained for each area or pixel of the image. When the luma or brightness component is analyzed on an area-by-area basis, the brightness value for an area can be represented by the brightness value of a pixel in the area or the average brightness value of a plurality of pixels in the area.

In the fifth step 78, the gain value as set by the imaging device 32, 44 for the overall image is also acquired from the image device 32, 44. This information may be acquired using a serial communication protocol that can query the imaging device 32, 44 for image control settings such as the overall gain setting for the image.

In the six step 80, a look-up table is preferably used to generate a subtraction factor for each area or pixel from the gain and luma values. Alternately, an equation may be used to calculate the subtraction factor from the luma and gain values. Preferably, the look-up table or equation is based on heuristics and empirical data. The subtraction factor is an indicator how much FPN should be subtracted from the image data to obtain the corrected FPN data. In general, an area or pixel with a high luma value would have a smaller subjection factor than one with a low luma value. In contrast, a high gain value would require a larger subtraction factor than a low gain value.

In the seventh step 82, the subtraction factor for each area or pixel may be used to modify the dark FPN value for the area or pixel by multiplying the dark FPN value with the subtraction factor for the area or pixel.

In the eighth step 84, the modified dark FPN values are then subtracted from the video image from the imaging device 32, 44 on an area-by-area basis or on a pixel-by-pixel basis. This process may be carried out repeatedly for every frame of the video image or for a selected number of frames. This process may be done dynamically in order to account for the rapid change in the brightness of the image.

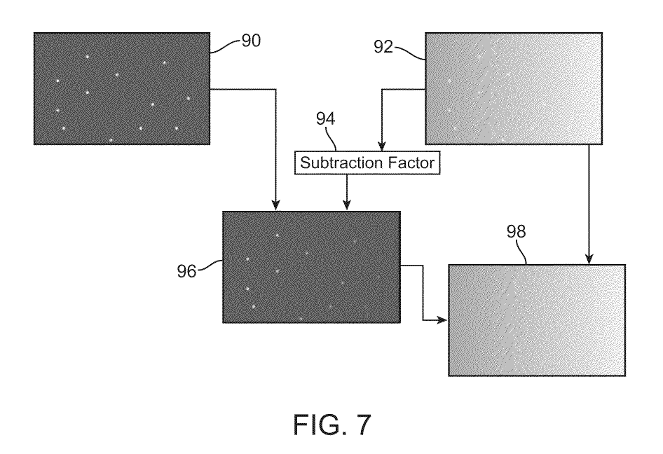

FIG. 7 shows various images generated by the above-described procedure. A dark FPN image 90 is acquired by the imaging device 32, 44 in a dark environment. As shown in FIG. 7, there is FPN (white dots) throughout this image 90. In the unprocessed output image 92 of the imaging device 32, 44, the dark area of the image has a higher level of FPN than the light area. Subtraction factor 94 for each pixel (or area) of the unprocessed output image 92 is obtained based on the brightness level of the pixel (or area) and the gain value. From the dark FPN image 90 and the subtraction factors 94, a modified dark FPN image 96 is obtained, which represents the corrected FPN level for each pixel (or area) in the unprocessed output image 92. The corrected FPN levels are subtracted from the unprocessed output image 92 to obtain the corrected output image 98.

As an example, the following is an illustration how the above-described procedure can be used in the colonoscopic procedure to reduce the FPN in the image captured by a retrograde imaging device. As an initial step of a colonoscopic procedure, a physician inserts the colonoscope into the patient's rectum and then advances it to the end of the colon. In order to achieve a greater viewing angle, the physician inserts a retrograde imaging device into the accessory channel of the endoscope and connects the video cable to the video processor, which includes the present invention's circuit/algorithm for FPN reduction. The video processor analyzes the image data received from the retrograde imaging device and reduces the FPN according to the above-described procedure. The physician may then carry out the procedure in a normal fashion. After the colonoscopic procedure is completed, the retrograde imaging device is retracted and the standard endoscope is removed.

In one alternate embodiment, the above-described procedure of the present invention can be modified to determine the subtraction factor for each area or pixel from not only the luma and gain values but also the operating temperature. In this embodiment, the lookup table or equation for the subtraction factor has three inputs: the luma and gain values and operating temperature.

In another alternate embodiment, the above-described procedure of the present invention can be modified to determine the subtraction factor for each area or pixel from the luma value alone without the gain value of the image. Alternatively, the procedure can be modified to determine the subtraction factor for each area or pixel from the gain value alone without the luma value.

In still another embodiment, the subtraction factor for each area or pixel can be determined from any one or more of the three parameters: the luma and gain values and operating temperature.

In yet another embodiment, in place of a dark FPN image used as a baseline for determining FPN, an FPN image, which is acquired by the imaging device 32, 44 with the imaging device 32, 44 in a given or known light conditions, can be used as a baseline for determining FPN. The given or known light condition may mean one or more of the relevant variables are known or given. As defined previously, the “relevant variables” are the variables that affect the FPN level of the area or pixel. These relevant variables include, but are not limited to, the brightness and color composition of the area or pixel, the operating temperature, the imaging device's voltage level and the gain of the image. This can be done as part of an initial factory calibration or periodically during the life of the imaging device 32, 44, such as every second during operation or at the beginning of each operation. This baseline FPN image is then stored in the memory of the imaging device 32, 44 such as an EEPROM or in the memory of the video processor 62. In this embodiment, the look-up table or equation for generating a subtraction factor for each area or pixel may have any one or more of the relevant variables as the dependent variables. These dependent variables can be obtained by analyzing the image data or from the imaging device. In the embodiment shown in FIG. 6, only the gain and luma values are the dependent variables. The thus obtained baseline FPN image and the look-up table or equation can be used to determine the “actual” FPN for an image area or pixel.

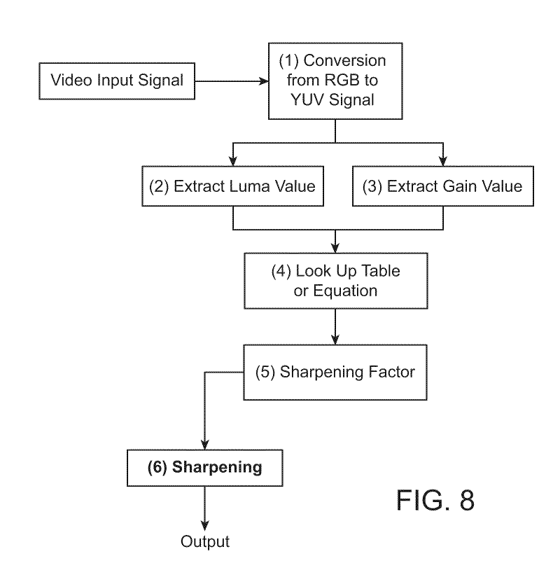

In a further alternate embodiment, as shown in FIG. 8, the above-described procedure of the present invention can be adapted for use with dynamic sharpening. Sharpening of an image can provide greater detail but can also lead to greater noise in the image particularly in darker areas of the image. The above-described procedure of the present invention can be used to reduce the noise created by dynamic sharpening. As a first step, the RGB signal from the imaging device is converted to a YUV signal. In the second step, the luma value of each pixel (or area) is acquired along with an overall gain value for the image. These two sets of values are acquired on a pixel-by-pixel basis (or on an area-by-area basis) and are then run through a look up table. Alternately, an equation can be used to ultimately lead to a sharpening factor. Given the sharpening factor, the overall image is passed through a standard sharpening algorithm such as a 3×3 convolutional filter to sharpen the image. Each pixel (or area) is subjected to the filter but only to a degree stipulated by the sharpening factor. As a result, bright areas of the image are sharpened more than dark areas of the image, providing greater details in the image and reducing extra noise.

In a still further alternate embodiment, dynamic sharpening can be combined with dynamic fixed pattern nose reduction. In such an embodiment, two sets of lookup tables and/or equations are employed in order to derive a sharpening factor and a subtraction factor. Appropriate steps are then taken to subtract the dark FPN image that has been scaled according to corresponding areas on the video image, while also sharpening appropriate areas.

1. A method for reducing a digital image's fixed pattern noise, comprising:
determining the amount of FPN in a digital image taken by a digital imaging device as a function of at least one of relevant variables on an area-by-area basis or on a pixel-by-pixel basis; and
modifying the digital image by the determined amount of FPN on an area-by-area basis or on a pixel-by-pixel basis.

2. The method of claim 1, wherein the relevant variables include a brightness level and color composition of the digital image in an area or pixel, an operating temperature, the imaging device's voltage level, and a gain of the digital image.

3. The method of claim 2, wherein the at least one of relevant variables includes only the brightness level of the image and the gain of the digital image.

4. The method of claim 2, wherein the at least one of relevant variables includes only the brightness level of the image.

5. The method of claim 2, wherein the at least one of relevant variables includes only the gain of the digital image.

6. The method of claim 2, wherein the at least one of relevant variables includes only the brightness level, operating temperature, and gain value of the image.

7. The method of claim 1, wherein the step of determining includes obtaining a baseline FPN image from the imaging device with the imaging device in a given or known light conditions.

8. The method of claim 7, wherein the baseline FPN image is stored in the imaging device's memory.

9. The method of claim 1, wherein the step of determining includes obtaining a dark FPN image from the imaging device with the imaging device in a dark environment.

10. The method of claim 9, wherein the dark FPN image is stored in the imaging device's memory.

11. The method of claim 10, wherein the step of determining includes determining a subtraction factor for each area or pixel using a look-up table having the subtraction factor as an output and the at least one of brightness level, operating temperature, and gain value of the image as one or more inputs.

12. The method of claim 11, wherein the step of determining includes determining the amount of FPN in the digital image by using the subtraction factor for each area or pixel to reduce the dark FPN value for this area or pixel, and wherein the dark FPN value is obtained from the memory of the imaging device.

13. The method of claim 10, wherein the step of determining includes determining a subtraction factor for each area or pixel using an equation having the subtraction factor at an independent variable and the at least one of brightness level, operating temperature, and gain value of the image as one or more dependent variable.

14. The method of claim 13, wherein the step of determining includes determining the amount of FPN in the digital image by using the subtraction factor for each area or pixel to reduce the dark FPN value for this area or pixel.

15. The method of claim 10, wherein the step of obtaining a dark FPN image includes obtaining the dark FPN image as part of an initial factory calibration.

16. The method of claim 10, wherein the step of obtaining a dark FPN image includes obtaining periodically during the life of the imaging device.

17. The method of claim 1, wherein the digital image is in YUV format, the method further comprising determining the brightness level from the luma component of the YUV format digital image.

18. The method of claim 1, wherein the digital image is in RGB format, the method further comprising
converting the RGB format digital image to a YUV format digital image, and
determining the brightness level from the luma component of the YUV format digital image.

19. A device for reducing a digital image's fixed pattern noise, comprising:
an input for receiving a digital image from a digital imaging device;
an output for sending a modified digital image to a display device;
a processor that includes one or more circuits and/or software for processing the digital image, wherein the processor determines the amount of FPN in a digital image taken by a digital imaging device as a function of at least one of relevant variables on an area-by-area basis or on a pixel-by-pixel basis and modifies the digital image by the determined amount of FPN on an area-by-area basis or on a pixel-by-pixel basis.

20. The device of claim 19, wherein the relevant variables include a brightness level and color composition of the digital image in an area or pixel, an operating temperature, the imaging device's voltage level, and a gain of the digital image.

21. The device of claim 20, wherein the at least one of relevant variables includes only the brightness level of the image and the gain of the digital image.

22. The device of claim 20, wherein the at least one of relevant variables includes only the brightness level of the image.

23. The device of claim 20, wherein the at least one of relevant variables includes only the gain of the digital image.

24. The device of claim 20, wherein the at least one of relevant variables includes only the brightness level, operating temperature, and gain value of the image.

25. The device of claim 19, wherein the processor determines the amount of FPN in the digital image by way of obtaining a baseline FPN image from the imaging device with the imaging device in a given or known light conditions.

26. The device of claim 25, wherein the baseline FPN image is stored in the imaging device's memory.

27. The device of claim 19, wherein the processor determines the amount of FPN in the digital image by way of obtaining a dark FPN image from the imaging device with the imaging device in a dark environment.

28. The device of claim 27, wherein the dark FPN image is stored in the imaging device's memory.

29. The device of claim 28, wherein the processor determines the amount of FPN in the digital image by way of determining a subtraction factor for each area or pixel using a look-up table having the subtraction factor as an output and the at least one of brightness level, operating temperature, and gain value of the image as one or more inputs.

30. The device of claim 29, wherein the processor determines the amount of FPN in the digital image by way of using the subtraction factor for each area or pixel to reduce the dark FPN value for this area or pixel.

31. The device of claim 28, wherein the processor determines the amount of FPN in the digital image by way of determining a subtraction factor for each area or pixel using an equation having the subtraction factor at an independent variable and the at least one of brightness level, operating temperature, and gain value of the image as one or more dependent variable.

32. The device of claim 31, wherein the processor determines the amount of FPN in the digital image by way of using the subtraction factor for each area or pixel to reduce the dark FPN value for this area or pixel.

33. The device of claim 28, wherein the processor obtains the dark FPN image as part of an initial factory calibration.

34. The device of claim 28, wherein the processor obtains the dark FPN image periodically during the life of the imaging device.

35. The device of claim 19, wherein the digital image is in YUV format, and wherein the processor determines the brightness level from the luma component of the YUV format digital image.

36. The device of claim 19, wherein the digital image is in RGB format, and wherein the processor converts the RGB format digital image to a YUV format digital image and determines the brightness level from the luma component of the YUV format digital image.

37. An endoscope system comprising:
the device of claim 19;
an endoscope including the digital imaging device and being connected to the input of the device; and
a displace device that is connected to the output of the device to receive and display the modified digital image.

38. The endoscope system of claim 37, wherein the digital imaging device is a retrograde-viewing auxiliary imaging device.

39. A method for sharpening a digital image, comprising:
determining the amount of sharpening needed to sharpen a digital image taken by a digital imaging device as a function of at least one of brightness level, operating temperature, and gain value of the image on an area-by-area basis or on a pixel-by-pixel basis; and
sharpening the digital image by the determined amount of sharpening on an area-by-area basis or on a pixel-by-pixel basis.

40. A device for sharpening a digital image, comprising:
an input for receiving a digital image from a digital imaging device;
an output for sending a sharpened digital image to a display device;
a processor that includes one or more circuits and/or software for shapening the digital image, wherein the processor determines the amount of sharpening needed to sharpen the digital image as a function of at least one of brightness level, operating temperature, and gain value of the image on an area-by-area basis or on a pixel-by-pixel basis and sharpens the digital image by the determined amount of sharpening on an area-by-area basis or on a pixel-by-pixel basis.
